(19) United States
(12) Patent Application Publication
(10) Pub. No.: US 2021/0082553 A1
(43) Pub. Date: Mar. 18, 2021

(54) A TOOL AND SYSTEM FOR GENERATION AND USE OF PERSONALIZED, INTERACTIVE LABORATORY REPORTS USING REAL-TIME REPORT

(71) Applicant: Venkata Satya Suresh ATTILI, Hyderabad (IN)

(72) Inventors: Venkata Satya Suresh ATTILI, Hyderabad (IN); Anuradha VUTUKURI, Hyderabad (IN)

(21) Appl. No.: 16/954,490

(22) PCT Filed: May 1, 2018
(86) PCT No.: PCT/IN2018/050268

§ 371 Date: Jun. 16, 2020

(30) Foreign Application Priority Data
Dec. 16, 2017 (IN) 201741045283

(51) Int. Cl. G16H 15/00; G16H 10/40; G16H 50/30; G16H 50/20

(52) U.S. Cl. CPC G16H 15/00; G16H 50/20; G16H 50/30; G16H 10/40

(57) ABSTRACT

A system and method for generating real-time cumulative personalized health screening report that provides more specific reporting pattern taking into account of interplay at the cellular/tissue and organ level. Further, the system and method for health screening and diagnosis is based on the outcomes in the present report, past diagnostic test readings and overall health condition besides trying to map with similar looking/matched population.

### TECHNICAL FIELD

The invention relates to a system and method for generation and use of personalized interactive laboratory reports using the real-time results and mapping with previous results of self and matched population in health care industry. More specifically, the invention relates to a system and method for health screening and diagnosis based on the outcomes in the present report, past diagnostic test readings and overall health condition besides trying to map with similar looking/matched population.

### BACKGROUND ART

Blood/laboratory testing relies on the chemical and physical characteristics of various blood components which often are represented/translated into numeric/digits, units, and acronyms. These shall remain alpha-numerical unless accompanied by interpretation and logical analysis. With the analysis this laboratory data shall explain about current health, risks for disease, and potential appropriate therapy and response to it.

Explaining lab tests is one of the most central diagnostic instruments used by doctors to identify if a patient in distress from any health condition. Whereas translating blood test results for the purposes of treatment and medical diagnosis has to be entrusted to physicians, gaining a basic understanding of how to figure out lab tests is significant. At the same time, it is equally important to understand the basics information, which goes ling way in seeking timely medical advice.

However it is not that easy to interpret laboratory results without domain knowledge. Familiarizing with domain knowledge for a common man is quite tough due to medical Jorgen. In many geographical regions, it's illegal for a laboratory to send test results directly to a patient making the agony worse. The blood may be patients but the information it contains is not. However this scenario is rapidly changing and with more and more nations allowing the access of lab results to patients, interpretation at common man level shall be the next bottle neck.

Even though there is bit of information like normal/abnormal, and literature regarding test, are printed often, the information is either too much limited or too complex to comprehend. Available method and systems at present merely provide the information for a specific laboratory test, whether the test is normal or abnormal. Very often leading to one of the two approaches; a dismissive approach wherein risk of missing a potential health condition is high or a paranoid approach where too many unnecessary hospital visits may happen with lot of anxiety associated and waste of resources.

Existing solutions, of which there are a few, have generally focused on maintenance of patient health records in an digital format and displaying them in a graphic format with no specific reference points for a specific screening test. Such systems and method focus on only one aspect of the patient health status at a given time thereby reducing the overall value of the medical advice.

At present there are many methods and systems each providing only a partial view of the patient's medical status with respect to a particular health issue? Such methods and systems do not provide a cumulative health screening report covering all aspects of the parent's health.

Therefore there is a need to provide for a method or system for generating real-time cumulative personalized health screening report which shall have to be interactive.

### SUMMARY OF INVENTION

Accordingly, in order to obviate the drawbacks in the existing state of art, the present invention discloses herein a system and method for generating real-time cumulative personalized health screening report. The system for generating the real-time cumulative personalized health screening report as described herein generates and uses personalized interactive laboratory reports using the real-time results and maps the same with the previous/earlier results of the self and/or with the matched population. The system as described herein provides more specific reporting pattern taking into account of interplay at the “cellular/tissue and organ level”, wherein a change in one laboratory parameter effects the remaining and thus helping to understand this interplay, by using a rule based system that upgrades itself on feed back mechanism and facilitates the individual to understand the same and seek timely intervention by improving awareness of the actual health condition. The health screening and diagnosis assisting system shall base its outcomes on the present report, past diagnostic test readings and overall health condition besides trying to map with similar looking/matched population.

The system as described herein provides 3 dimensional pictorial representation of human anatomy where the interpreter will be able to understand as to “what could be the impact of variation of a lab report(s) (both normal and abnormal raged) on various systems/physiologies”, in an interactive manner and accordingly generates recommendations to concerned medical advisors for each aspect of the patients health including indication of potential health risks based on the summary of overall risk level for all aspects of a patient's health. The output/reports could be as informative to patients as they are to the physicians. Appropriate context, animation, color, and literature based abstract (from the available medical literature) allows it possible to make sense of these reports (not just alpha numerical any more). This enables the patients to interpret the report and can become participants in their own health.

The system as described herein is also capable of advising timely medical intervention based on the summary of overall risk level for all aspects of a patient's health and accordingly the system is capable of suggesting lifestyle changes based on the summary of overall risk level for all aspects of a patient's health. The system according to the invention described herein comprises of rule based algorithms supported by big data analytics and machine learning (AI), with a user friendly graphic user interface, which converts the complex medical/diagnostic reports into simple and easily understandable/interpretable reports.

The invention also provides a method that summarizes the overall risk level for all aspects of a patient's health and generating detailed interpretative information for each specific screening test for each aspect of the patient's health. The method as described herein comprises the step of receiving screening test result for a specific aspect of patient's health, reporting it in the conventional form, retrieving past results of all screening tests of the patient's overall health, generating a real-time cumulative personalized health screening report and representing real-time cumulative personalized health screening report in a graphical interface.

### DESCRIPTION OF EMBODIMENTS

According to one embodiment of the present invention, the graphical nature and data, change in blood reports over time, comparison across and within reports to provide real-time cumulative personalized health screening report information to the patient in a manner that can be easily interpreted to seek timely medical intervention. In order to interpret the screening results, the system as described herein makes use of a standard reporting pattern represented in graphical matrix data time points for easy recognition of the drifts. The system according to the invention summarizes the overall risk level for all profiles along with detailed interpretive information on each profile and test from the recommendations of various authorities.

The interpretation reports are representative of laboratory results, calculated based on uniquely designed and scientifically validated algorithms, which are easy to understand with highest accuracy.

The method of the present invention comprises the step of receiving blood/laboratory test result for a specific aspect of patient's health, reporting it in the conventional form, retrieving past results of all screening tests of the patient's overall health, and matched population and generating a real-time cumulative personalized health report.

The method developed multiple human anatomical models with super-imposed ultras structural characters (at cellular level) and incorporated physiological functions to the same.

Once the information of the labs is fed, the rule based algorithm puts across various organ (anatomical/physiological) entities and maps the lab results to corresponding sections, followed by a color coding with red to green (shades based on how far it is from the normal range).

This is further followed by a basic representation of the other parameters with ranges and the representation shall be in such a way that any change in one value affects all other values in an intelligent manner.

### EXAMPLES

Example 1—With a fall in the serum Iron level, it gives a probable prediction of the

Hemoglobin, from where all the errors could be (eg. blood loss, poor absorption etc.), or based on the ferritin and transferring, what might be going on inflammatory level or at a liver level. As the complexity increases, with more information, the differential diagnosis/medical possibilities shall narrow down based on algorithm.

The steps involved include the following

Performing the test

Report in conventional form

Applying the proven matrix to interpret the possible condition

Using algorithm to fix where the patient fits into

Coding the outcome with appropriate color [ High risk-red, normal green etc]

The color coded results are highlighted in the graphical format

The comparison with previous results [ if any] are superimposed with final outcome calculator [ again algorithm based]

Representing the human anatomy

Popping the relevant organ, to which the test belong to

Mapping the lab results with color code to the all organs that influences the particular lab results

Popping of Cursor

Movement (increase or decrease) shall lead to the effect of a given lab result on various other organs as represented in 3 D image

Literature based text shall follow as foot note

1. A system for generating real-time cumulative personalized health screening report comprising of

2. generating and using personalized interactive laboratory reports using the real-time results,

3. mapping the same with the previous/past results of the self and/or with the matched population, and

4. providing specific reporting pattern taking into account of interplay at the cellular/tissue and organ level.

5. The system as claimed in claim 1, wherein the reporting pattern provides 3 dimensional pictorial representation of human anatomy where the interpreter will be able to understand as to what could be the impact of variation of a lab report(s) (both normal and abnormal raged) on various systems/physiologies”, in an interactive manner.

6. The system as claimed in claim 1, wherein the reporting pattern generates recommendations to concerned medical advisors for each aspect of the patients health including indication of potential health risks based on the summary of overall risk level for all aspects of a patient's health.

7. The system as claimed in claim 1, wherein the system comprises of rule based algorithms supported by big data analytics and machine learning (AI), with a user friendly graphic user interface, which converts the complex medical/diagnostic reports into simple and easily understandable/interpretable reports.

8. A method for summarizing the overall risk level for all aspects of a patient's health and generating detailed interpretative information for each specific screening test for each aspect of the patient's health according to the system of claim 1, the method comprising the steps of

9. receiving screening test result for a specific aspect of patient's health, reporting it in the conventional form,

10. retrieving past results of all screening tests of the patient's overall health, and

11. generating a real-time cumulative personalized health screening report and representing real-time cumulative personalized health screening report in a graphical interface.
